(19) United States
(12) Patent Application Publication
(10) Pub. No.: US 2011/0091559 A1
(43) Pub. Date: Apr. 21, 2011

(54) COMPOSITIONS COMPRISING ADJUVANT, MACROLIDE AND PROTEINACEOUS ANTIGEN AND METHODS OF USE THEREOF

(75) Inventors: Nahla Fattohi, Robbinsville, NJ (US); Terry Kaleung Ng, Fort Dodge, IA (US)

(73) Assignee: Wyeth LLC, Madison, NJ (US)

(21) Appl. No.: 12/898,009

(22) Filed: Oct. 5, 2010

Related U.S. Application Data
(60) Provisional application No. 61/249,454, filed on Oct. 7, 2009.

(51) Int. Cl. A61K 9/113; A61K 39/00; A61K 39/002

(52) U.S. Cl. 424/489; 424/265.1; 424/191.1

(57) ABSTRACT

The invention is directed to compositions comprising an oil adjuvant, a macrocyclic lactone effective for the prevention or control of parasitic infection in a warm-blooded animal and an immunizing amount of at least one immunogenic polypeptide and methods of use thereof.

### CROSS-REFERENCE TO RELATED APPLICATIONS

This application claims the benefit of U.S. Provisional Application No. 61/249,454, filed Oct. 7, 2009, the entire contents of which is incoporated herein by reference.

### BACKGROUND OF THE INVENTION

Helminthiasis is a widespread disease found in many animals and is responsible for significant economic losses throughout the world. Among the helminths most frequently encountered are the groups of worms referred to as nematodes and trematodes. Trematodes are found in the intestinal tract, heart, lungs, blood vessels and other body tissues of animals and are a primary cause of anemia, weight loss and malnutrition in the infected animals, and cause economic losses. They do serious damage to the walls and tissue of the organs in which they reside and, if left untreated, may result in death to the infected animals.

The trematode, Fasciola hepatica (liver fluke) is a frequent cause of disease in ruminants. Animals are infected when they consume mollusks, e.g., snails, harboring the infectious stage of the parasite. The effects of liver fluke are referred to as fascioliasis, and include anemia, weight loss and sub-mandibular edema and, on occasion, diarrhea. A serious consequence of the liver damage caused by fascioliasis is that latent Clostridium novyi spores can be activated by the low oxygen conditions in the damaged tracts the parasite forms in the liver—this can lead to “black disease,” caused by Clostridium novyi type B or immune-mediated hemolytic anemia (IMHA) leading to hemoglobinuria caused by Clostridium novyi type D.

Macrolide compounds including macrocyclic lactones, such as milbemycin compounds, such as moxidectin, milbemycin D, milbemycin oxime and nemadectin, avermectin compounds such as abamectin, ivermectin and doramectin, and mixtures thereof are useful for the prevention and control of helminthiasis and infection by acarids and arthropod endo- and ectoparasites in warm-blooded animals. Subcutaneous injection of aqueous compositions is one of the preferred methods for administering those compounds. Vaccines are used to protect warm-blooded animals from a variety of diseases and are also administered by subcutaneous injection.

It would be highly advantageous to be able to co-administer a vaccine with a macrolide compound. This would significantly reduce the workload, for example, in treating livestock, and could lead to significant economic advantages. In many parts of the world it is common to vaccinate livestock against infestation with parasites such as ticks and nematodes. Following vaccination, a significant time lag occurs before a significant reduction in parasite load is observed, due to the time taken for induction of an immune response against the parasite. Because of this time lag, farmers often use an additional antiparasitic drug to reduce parasite infestation during the time lag.

Due to the large size and complexity of helminth parasites, it is difficult to vaccinate using killed whole organism or organism extracts. It is thus preferable to vaccinate against helminth parasites vaccines using so-called “subunit vaccines,” comprising one or more partially or fully purified helminth proteins, protein fragments or derivatives thereof as antigen.

Stable vaccine compositions containing both a macrolide compound and protein antigens, however, are difficult to formulate, however, due to lack of stability of one or both of the antigen or the macrolide compound. Many macrolide compounds are partially or completely insoluble, or are unstable in, aqueous solution. Moreover, even when possible to formulate aqueous injectable compositions of macrolide compounds, such compositions often contain dispersing agents that are known to interact with proteins and affect the permeability of the membrane of cells. Such interaction can denature or otherwise disrupt proteins such as antigens. Additionally, injectable compositions are most effective for vaccination when injected in the form of an emulsion. An additional variable in forming effective stable vaccine compositions containing both a macrolide compound and antigens is thus the stability of the emulsion.

Accordingly, the macrolide compounds cannot easily be admixed with protein antigens in a vaccine for a “single shot” administration, and separate administration of the vaccine and antiparasitic drug is required, leading to increased work and higher cost.

Formulations that can be used to simultaneously administer a subunit vaccine and one or more macrolide compounds are thus highly desirable, but are difficult to formulate. The inventors of the present invention have discovered compositions comprising an adjuvant, macrolide compounds and proteineceous antigen that are useful in methods for protecting or controlling helminthiasis, infection by acarid and arthropod endo- and ectoparasites and bacterial and viral disease in warm-blooded animals.

### BRIEF SUMMARY OF THE INVENTION

The present invention is directed to compositions that are useful for prophylaxis and for treating disease in mammals. The compositions are preferably stable, may be stored for prolonged periods of time without loss of antigen and macrolide potency and are most preferably maintained as emulsions.

In certain aspects, the invention provides a composition comprising an oil adjuvant, a macrocyclic lactone effective for the prevention or control of parasitic infection in a warm-blooded animal and an immunizing amount of a immunogenic polypeptide.

In certain aspects, the invention provides composition comprising a macrocylic lactone effective for protecting or controlling helminthiasis, or infection by an acarid or arthropod endo- or ectoparasite in said warm blooded animal.

In certain aspects, a macrocyclic lactone is a milbemycin compound, an avermectin compound or a combination thereof. In one embodiment, the milbemycin compound is, for example, moxidectin, nemadectin, milbemycin D or milbemycin oxime or a combination thereof. In a further embodiment, the avermectin compound is, for example, abamectin, doramectin, ivermectin, selamectin or eprinomectin, emamectin or a combination thereof.

In certain aspects, the invention provides compositions in the form of an oil in water emulsion. For example, in one embodiment, the adjuvant composition includes SP Oil, Emulsigen, Montanide™, sulfolipo-cyclodextrin in squalene in water emulsion (SL-CD) or combinations thereof. The oil adjuvant can include a metabolizable oil, a non-metabolizable oil or a combination thereof. Preferably, the oil adjuvant includes a metabolizable oil. In a preferred embodiment, the adjuvant composition includes both SP Oil and SL-CD.

In certain aspects the invention provides compositions further comprising one or more of a dispersing agent, water soluble organic solvent, and/or preservative.

In certain aspects, compositions comprise an immunogenic polypeptide that is derived from a parasite of said warm blooded mammal, such as a nematode or trematode, such as for example a Fasciola species, e.g., Fasciola hepatica.

In certain aspects, the immunogenic polypeptide is derived from a Fasciola cathepsin protease, glutathione-S-transferase, dipeptidyl peptidase, excretory/secretory (E/S) product, peroxiredoxin, β-tubulin, α-tubulin, or haemoprotein, fragment thereof, or a polypeptide having 80% or 90% identity thereof.

In certain embodiments, the invention provides methods of using the aforementioned compositions for the prevention or control of a parasitic infection in warm blooded mammal by administering an effective amount of the compositions to a warm blooded mammal.

### DETAILED DESCRIPTION OF THE INVENTION

The present invention provides a combination of vaccine and pharmaceutical compositions that are useful for prophylaxis and for treating disease in mammals. The terms “composition”, vaccine composition” and “vaccine and pharmaceutical composition” may be used interchangeably herein. However, each of these terms is intended to mean a composition that is a mixture of a vaccine and a pharmaceutical. The compositions are stable, may be stored for prolonged periods of time without loss of antigen and macrolide potency and are maintained as emulsions.

In certain embodiments, the present invention thus provides a stable vaccine composition comprising a macrolide compound as hereinafter defined, an oil adjuvant, and at least one immunogenic polypeptide as antigen. In one embodiment, the at least one immunogenic polypeptide is derived from a Fasciola cathepsin protease, a Fasciola peroxiredoxin or a combination thereof. Vaccine compositions may, additionally, comprise one or more of a water soluble organic solvent, a dispersing agent, and a preservative. In certain embodiments, stable vaccine compositions thus comprise a macrolide compound as hereinafter defined, an adjuvant, a water soluble organic solvent, and at least one antigen. In certain embodiments, stable vaccine compositions comprise a macrolide compound as hereinafter defined, an oil adjuvant, a water soluble organic solvent, at least one antigen and a preservative. In preferred embodiments, the vaccine compositions are present in the form of an oil-in-water emulsion, comprising adjuvant particles in an aqueous base.

The aforementioned vaccine compositions are useful in methods for protecting or controlling helminthiasis, infection by acarid and arthropod endo- and ectoparasites and disease in warm-blooded animals.

Macrolide compounds useful in the invention include, for example and without limitation, macrocyclic lactone compounds. Macrocyclic lactones suitable for use in compositions and methods of the invention include, for example and without limitation, milbemycins such as moxidectin, nemadectin, milbemycin D or milbemycin oxime, or the like, preferably moxidectin. Macrocyclic lactone compounds include the compounds disclosed in U.S. Pat. No. 5,989,566, which is incorporated herein by reference.

Macrocyclic lactones suitable for use in compositions and methods of the invention also include, for example and without limitation, avermectin compounds such as abamectin, doramectin, ivermectin, selamectin, emamectin, or eprinomectin. Preferred avermectin compounds are abamectin, ivermectin, doramectin. Doramectin and a method for its preparation are described in U.S. Pat. No. 5,089,480, incorporated herein by reference. Avermectin compounds are also described in U.S. Pat. Nos. 4,199,569 and 4,310,519, incorporated herein by reference.

Additional suitable macrocyclic compounds include, but are not limited to, those described in U.S. Pat. No. 5,019,589; 4,886,828; 5,108,992; 5,030,650 and 5,055,486, incorporated herein by reference. Other suitable avermectins and milbemycins are described in EP 0750907A2, EP 0413538A1, WO96/37178, EP 0525307, and U.S. Pat. No. 4,853,372 and No. 4,389,397.

Macrocyclic lactone compounds are typically included in vaccine compositions at about 0.01 to about 2.0% w/v, preferably 0.1 to about 1.0% w/v, 0.1% to about 10.0% w/v, preferably about 0.5% to about 5% w/v, and more preferably from about 0.5% to about 2% w/v, and about 0.5 to about 3.0% w/v, more preferably about 1.0 to about 2.5% w/v. In certain embodiments, macrocyclic lactone compounds are present in vaccine compositions at about 1% w/v. The effective amounts may vary according to the potency of the compounds, the method of application, the host animal, the target parasite, the degree of infestation, or the like. For injectable vaccine compositions for administration to large animals such as swine, sheep, horses or cattle, the above amounts moxidectin may be suitable. Preferred amounts of moxidectin for use in compositions and methods as described are, without limitation 0.5% to about 2% w/v, and about 0.5 to about 3.0% w/v, more preferably about 1.0 to about 2.5% w/v and most preferably about 1% w/v.

Macrolide compounds may be formulated with the use of dispersing agents. Dispersing agents useful in the compositions and methods of the present invention include polyethylene oxide sorbitan mono-oleates such as polyoxyethylene (20) sorbitan mono-oleate (TWEEN™ 80, Harcros Chemicals), polyoxyethylene alcohols such as laureth 9 and cetomacrogol 1000, sodium lauryl sulfate, dioctyl sodium sulfosuccinate, polyethylene glycols, and α-hydro-ω-hydroxypoly(oxyethylene) poly(oxypropylene)poly(oxyethylene)block copolymers with polyethylene oxide sorbitan mono-oleates such as polyoxyethylene (20) sorbitan mono-oleate being preferred.

As used herein the term “adjuvant” refers to any component which improves the body's response to a vaccine. Further, as used herein, the term “oil adjuvant” refers to an adjuvant that includes an oil, such as a non-metabolizable mineral oil, a metabolizable oil, or a combination thereof. The non-metabolizable oils include mineral oils, such as white mineral oil, and light mineral oil. The metabolizable oils include vegetable oils, fish oils and synthetic fatty acid glycerides. In one embodiment, the adjuvant further includes a surfactant/emulsifier. Preferably, the adjuvant is an oil-emulsion, such as any water-in-oil (w/o) emulsion, oil-in-water (o/w) emulsion, and w/o/w emulsion which can be administered to living animals without unacceptable side-effects. In a preferred embodiment, the oil adjuvant is an o/w emulsion. The term “oil-in water emulsion” means an emulsion in which small droplets of oil are suspended in a continuous water phase. Usually, an oil-emulsion is composed of an aqueous phase, which can be made up of water, saline or a buffer (e.g., Phosphate Buffered Saline), an oil phase and one or more emulsifiers, which components are extensively mixed by known techniques until a stable emulsion is obtained. As is known in the art, the preparation of an o/w emulsion or a w/o emulsion, respectively, involves the appropriate choice of a suitable type of emulsifying agent, having regard to the relative proportions of the oil and water phases and their exact nature. The type of emulsion which the emulsifier is likely to promote is indicated by its relative affinity for oil and water, which is known as its hydrophilic-lipophilic balance (HLB). Generally, emulsifiers with an HLB of about 3-6 are required for the production of w/o-type emulsions. Suitable emulsifiers for o/w-type emulsions are usually found in the range of 10-18 (HLB). It is also general practice to combine two or more emulsifiers in such a way that a desired HLB value is obtained. Details concerning the production of pharmaceutical oil-emulsions can be found, for example, in: “The Theory and Practice of Industrial Pharmacy” (eds.: Lachman, L. et al., Lea & Febiger, Philadelphia, U.S.A., 1970, Chapter 16), “Remington's Pharmaceutical Sciences” (ed.: Gennaro, A. R., Mack Publishing Company, Easton, U.S.A., 1990, 18^{th} edition, “Bio-emulsifiers”, Zajic, J. E. et al. (in CRC Critical Reviews in microbiology, 1976, 19-66).

The adjuvant will typically comprise about 0.1 to 50% vol/vol of the vaccine formulation of the invention, more preferably about 1 to 50% of the vaccine, and even more desirably about 1 to 30% thereof. Amounts of about 10 to 25% may be even more preferred.

Suitable adjuvants can include immunostimulating oils such as certain metabolizable oils. Oils suitable for use in the composition of the invention include oil emulsions, e.g., SP Oil (hereinafter described), Emulsigen (MPV Laboratories, Ralston, NZ), Montanide™, such as ISA 50V, ISA 206 and IMS 1312, (Seppic SA, Paris, France), sulfolipo-cyclodextrin in squalane in water emulsion (SL-CD; described in Romera et al., Vaccine, 2000, 19:132-41) as well as peanut oil and other vegetable-based oils, squalane (shark liver oil) or other oils which can be shown to be suitable as an adjuvant in veterinary vaccine practice. Also suitable are adjuvant mixtures comprising one or more adjuvant, e.g., one or more of the aforementioned adjuvants. Further examples of suitable adjuvants are described herein.

In a preferred embodiment, the oil adjuvant includes a metabolizable oil. Metabolizable oils are non-mineral oils. Non-mineral oils are quickly metabolized and removed from the injection site, thus they have very few side-effects. Any metabolizable oil, particularly from an animal, a fish or vegetable source may be used herein, if desired in refined or chemically modified forms. Examples of useful vegetable oils include peanut oil, soybean oil, sesame oil, coconut oil, olive oil, cotton seed oil, safflower oil, sunflower oil, corn oil, rapeseed oil, grapeseed oil, almond oil, black current seed oil, jojoba oil, wheat germ oil, canola oil, or a triglyceride oil. Most fish contain metabolizable oils which may be used herein, such as cod liver oil and shark liver oil. In addition, terpenoid derivatives from fish oils, such as squalene mabe be used for the preparation of the adjuvant. Combinations of metabolizable oils can also be used in the compositions of the present invention.

The adjuvant compositions and vaccine compositions include a buffered aqueous base. The aqueous base may include about 1% to about 80% v/v, about 5% to about 80% v/v, about 5% to about 50%, about 10% to about 75% v/v, about 15% to about 60% v/v, or about 30% to about 60% v/v of an adjuvant composition. A preferred buffered aqueous base is PBS, e.g., 0.01 M PBS.

As described above, the adjuvant can be a Montanide™ Incomplete Seppic Adjuvant (Montanide ISA). Montanide ISA adjuvants are a group of oil/surfactant-based adjuvants where a non-metabolizable and/or metabolizable oil is combined with surfactants (available from Seppic, Belgium). Non-limiting examples of suitable Montanide ISA adjuvants include Montanide ISA-51, Montanide ISA 50, Montanide ISA 70, Montanide ISA 206, Montanide ISA 708, Montanide ISA-720, Montanide ISA763A, Montanide ISA207, Montanide ISA 264, Montanide ISA 27, Montanide ISA 35, Montanide ISA 740, Montanide ISA 773, Montanide ISA 266, Montanide ISA 267, and Montanide ISA 28. In one embodiment, the Montanide ISA adjuvant is a metabolizable, non-mineral oil based adjuvant, such as Montanide ISA 708, Montanide ISA-720, Montanide ISA 763A, Montanide ISA 207, Montanide ISA 264, Montanide ISA 27 and Montanide ISA 35.

As an adjuvant, SP Oil is preferred. The SP oil can be used together with other adjuvants, if desired. For example, in one embodiment, the adjuvant composition includes both SP oil and sulfolipo-cyclodextrin in squalane in water emulsion (SL-CD). As used in the specification and claims, the term “SP Oil” designates an oil emulsion comprising a polyoxyethylene-polyoxypropylene block copolymer, squalane, polyoxyethylene sorbitan monooleate and a buffered salt solution. In general, the SP Oil emulsion will comprise about 1 to 3% vol/vol of block copolymer, about 2 to 6% vol/vol of squalane, more particularly about 3 to 6% of squalane, and about 0.1 to 0.5% vol/vol of polyoxyethylene sorbitan monooleate, with the remainder being a buffered salt solution.

When utilized, immunogenically stimulating amounts of SP Oil as adjuvant in the vaccine composition of the invention may vary according to the immunogenically active component, the degree of potential infectious exposure, method of administration of the vaccine composition, the age and size of the animal, or the like. In general, amounts of about 1% to 50% vol/vol, preferably about 5% to 50% vol/vol, and more preferably about 15% to 30% vol/vol of SP Oil are suitable.

In addition, the adjuvant may include one or more wetting or dispersing agents in amounts of about 0.1 to 25%, more preferably about 1 to 10%, and even more preferably about 1 to 3% by volume of the adjuvant. Particularly preferred as wetting or dispersing agents are non-ionic surfactants. Useful non-ionic surfactants include polyoxyethylene/polyoxypropylene block copolymers, especially those marketed under the trademark PLURONIC™ and available from BASF Corporation (Mt. Olive, N.J.). Other useful nonionic surfactants include polyoxyethylene esters such as polyoxyethylene sorbitan monooleate, available under the trademark TWEEN 80™. It may be desirable to include more than one, e.g., at least two, wetting or dispersing agents in the adjuvant as part of the vaccine compositions.

Pharmacologically acceptable carriers suitable for use in the vaccine composition of the invention may be any conventional liquid carrier suitable for veterinary pharmaceutical compositions, preferably a balanced salt solution or other water-based solution suitable for use in tissue culture media. Other available carriers may also be utilized.

Additional excipients available in the art may also be included in the vaccine and pharmaceutical compositions according to the various embodiments heretofore described. For example, pH modifiers and, metal chelators may be utilized.

The components of the vaccine composition of the invention as heretofore described, including the carrier, may be combined together using available techniques.

In a preferred embodiment of the invention, the inventive vaccine composition may be formulated in dosage unit form as heretofore described to facilitate administration and ensure uniformity of dosage. Formulation may be effected using available techniques, such as those applicable to preparations of emulsions.

Stable vaccine compositions are prepared, for example, by mixing a macrolide composition comprising a macrolide compound dissolved in one or more solvents and a dispersing agent with an adjuvant composition comprising an oil-in-water emulsion of at least one adjuvant and at least one immunogenic polypeptide in an aqueous base. Vaccine compositions are preferably formed at a temperature of about temperature 21° C. or higher, preferably at room temperature (25° C.).

Vaccine compositions typically comprise about 0.1% to 10% of macrolide compound on a weight basis and preferably about 0.5% to 5% of the macrolide compound, e.g., about 1% of the macrolide compound; about 5% to about 30% of dispersing agent on weight basis, and preferably about and preferably about 10% to about 25% of dispersing agent on weight basis, e.g., about 20% of the dispersing agent; about 1% to about 40% of solvent on weight basis, and preferably about 10% to about 25% of solvent on a weight basis, e.g., about 20%; and about 5% to about 50% of adjuvant on a volume/volume basis, and preferably about 10% to about 30% of adjuvant on a volume/volume basis and more preferably from about 15% to about 25% of adjuvant on a volume/volume, e.g. about 15% or about 25%, and immunogenic polypeptide, as described below, in an amount of about 0.01 to about 10 mg/ml, about 0.01 to about 5 mg/ml, about 0.05 mg/ml to about 10 mg/ml, or about 0.05 mg/ml to about 5 mg/ml. In certain embodiments, immunogenic polypeptide is present in adjuvant compositions in the amount of about 0.1 mg/ml.

In a preferred embodiment, stable vaccine compositions are prepared by mixing a macrolide composition comprising moxidectin dissolved in one or more solvents and a dispersing agent with an adjuvant composition comprising an oil-in-water emulsion of at least one adjuvant and at least one immunogenic polypeptide in an aqueous base. Further preferred are stable vaccine compositions wherein the macrolide composition is formulated by dissolving moxidectin in a first solvent to form a first moxidectin composition and then mixing the first moxidectin composition with a second solvent and a dispersing agent to form a second moxidectin composition and mixing the second moxidectin composition with the adjuvant composition. In preferred embodiments, said first solvent is benzyl alcohol, said second solvent is propylene glycol and said dispersing agent is polysorbate 80.

Vaccine compositions may include one or more preservative (e.g., without limitation, anti-oxidants and anti-microbials), which may be present in one or both of the macrolide composition or the adjuvant composition or added separately. Preservatives suitable for use in the present invention include thimerosal ([(o-carboxyphenyl)-thio]ethylmercury sodium salt), formaldehyde, phenol, propylene glycol, glycerol, esters of p-hydroxybenzoic acid, benzoic acid and sodium benzoate. Preferred preservatives are is butylated hydroxytoluene (BHT) and thimerosal.

As used herein, the term “w/w” designates weight/weight, “w/v” designates weight/volume, “v/v” designates volume/volume, and the term “mg/kg” designates milligrams per kilogram of body weight.

An immunogenic polypeptide for use in compositions and methods described herein can be any polypeptide that is capable of generating a protective immune response in the host mammal. The skilled artisan will appreciate that a large number of such immunogenic polypeptides are known in the art. The term “polypeptide” is employed here to denote any peptide having two or more amino acids linked by peptide bonds. The immunogenic polypeptide may contain a single polypeptide chain, or may contain multiple polypeptide chains linked together, for example, by disulfide bonds or by other chemical cross-links. Polypeptides chains containing less than about 50 amino acids produce only a weak immune response due to their small size. Typically, these small polypeptides are linked to a larger carrier protein, such as serum albumin, to enhance the immune response against the immunogenic polypeptide. Methods of linking polypeptides to carrier proteins are well known in the art. Preferred single chain polypeptides for the present invention typically contain over 75 amino acids and, more typically, over 150 amino acids.

As used herein the terms “immunogenic” or “immunogenically active” designates the ability to stimulate an immune response. One of ordinary skill in the art will appreciate that an immune response may be either a humoral response that stimulates the production of antibodies, particularly humoral antibodies, or a cell-mediated response, or a combination of humoral and cell-mediated responses. For example, the ability to stimulate the production of circulating or secretory antibodies or the production of a cell-mediated response in local mucosal regions, (e.g., intestinal mucosa), peripheral blood, cerebral spinal fluid or the like.

The effective immunizing amount of the immunogenically active component may vary and may be any amount sufficient to evoke an immune response and provide immunological protection. Immunologically active components include, for example and without limitation, killed or inactivated whole or subunit virus cells or antigen or DNA cells derived therefrom or a mixture thereof. Preferred immunologically active components are proteinaceous substances such as, for example and without limitation, proteins or protein fragments.

Proteins and protein fragments are preferably purified from components that are to be included in vaccine compositions in reduced amounts or to be substantially excluded from vaccine compositions. One of ordinary skill in the art will appreciate that protein or protein fragments may thus be partially purified, highly purified or substantially completely purified from components that are to be reduced or substantially excluded from vaccine compositions. In preferred embodiments, proteins or protein fragments are highly or substantially completely purified.

The term “polypeptide” is used in its broadest sense and, for convenience. includes peptides, polypeptides, proteins, glycoproteins and fusion molecules. A polypeptide is generally in isolated form or recombinant or synthetic form. When in isolated form, the polypeptide has undergone at least one purification or isolation step. Preferably, however, the isolated molecule is in a form suitable for use in a vaccine and/or represents at least 5%, preferably at least 20%, more preferably at least 35%, still more preferably at least 55-60%, even more preferably at least 75-80% or yet even more preferably at least 90-100% of a composition relative to other components. The percentage content is conveniently measured by, for example, weight, activity, antibody reactivity or other like means.

It will be appreciated that once purified polypeptides have been obtained they can be admixed with one or more further purified antigenic proteins, to form a polyvalent vaccine.

The present invention extends to non-naturally occurring (i.e. synthetic) derivative of the subject polypeptides including derivatives which incorporate non-naturally occurring amino acid residues or chemical equivalent, homologues or analogues of naturally occurring amino acid residues.

Advantageously, the immunogenic polypeptide is derived from an organism that causes disease or other adverse health effects in humans or other mammals. The disease-causing organism may be a bacterium, virus, or other microbe, or may be a parasitic organism, such as a tick or insect or parasitic worms, such as trematodes or nematodes.

The term “derived” is employed here, in relation to the immunogenic polypeptides of the present invention, to denote those obtained by isolation and purification from a disease-causing organism, such as a tick, insect or trematode or nematode life stage which expresses the polypeptide, as well as antigens obtained by manipulation and expression of nucleotide sequences prepared from the organism. “Derived” also encompasses polypeptides produced from nucleotide sequences that encode the polypeptide, including genomic DNA, mRNA, cDNA synthesized from mRNA, and synthetic oligonucleotides. It further encompasses synthetic polypeptides prepared on the basis of the known amino acid sequences of the immunogenic polypeptides produced by the disease-causing organism. The polypeptide may have the same amino acid sequence as all or part of a protein expressed in the disease-causing organism, or the amino acid sequence may be modified to enhance the immune response against the organism. In a preferred embodiment, “derived” refers to a fragment of a polypeptide that is at least 8 amino acids in length, at least 20 amino acids in length, at least 30 amino acids in length. In an additional embodiment, “derived” refers to a polypeptide that is at least 80% or at least 90% identical to a reference polypeptide or at least 80% or at least 90% identical to a fragment of a reference polypeptide that is at least 8 amino acids in length, at least 20 amino acids in length, at least 30 amino acids in length. Determination of percent identity between or among polypeptides may be accomplished using methods that are well known in the art such as, for example and without limitation, the BLAST program (Altschul et al., J. Mol. Biol., 1990, 215:403-410), a version of which is publicly available from, e.g., the National Center for Biotechnology Information (NCBI).

Methods of modifying amino acid sequences to enhance an immunogenic response are well known in the art. The polypeptide may or may not be glycosylated, and/or may contain other post-translational modifications.

The at least one immunogenic polypeptide is typically present in adjuvant compositions in the amount of about 0.01 to about 10 mg/ml, about 0.01 to about 5 mg/ml, about 0.05 mg/ml to about 10 mg/ml, or about 0.05 mg/ml to about 5 mg/ml. In certain embodiments, the at least one immunogenic polypeptide is present in adjuvant compositions in the amount of about 0.1 mg/ml.

Without being bound by theory, the adjuvant compositions described herein and the vaccine compositions prepared using such adjuvant composition are believed to be present in the form of oil in water microemulsions wherein immunogenic polypeptide is present on the surface of adjuvant particles. Such polypeptide-adjuvant particles are believed to particularly suited for uptake of immune cells, facilitating generation of an immune response, and particularly most effective in enhancing a cellular mediated response.

In preferred embodiments, the adjuvant compositions described herein and compositions comprising the adjuvant compositions, i.e., composition comprising an adjuvant, a macrolide and an immunogenic polypeptide, are present in the form of oil in water emulsions, more preferably microemulsions. Such emulsions may comprise particles having an average particle size of, for example and without limitation, less than about 500 μm, less than about 350 μm, less than about 200 μm, less than about 100 μm, less than about 50 μm, less than about 25 μm or less than about 10 μm, or, for example and without limitation, an average particle size in the range of about 10 μm to about 500 μm, about 25 μm to about 350 μm, about 100 μm to about 200 μm, about 25 μm to about 100 μm, about 25 μm to about 200 μm, about 50 μm to about 100 μm or about 50 μm to about 100 μm.

Homeothermic animals suitable for treatment using the composition and method of the present invention include, for example and without limitation, swine, cattle, sheep, horses, goats, camels, water buffalos, donkeys, mules, rabbits, fallow deer, reindeer, minks, chinchillas, raccoons, chicken, geese, turkeys, ducks, dogs, cats, or the like, preferably dogs, cats, swine, cattle, horses or sheep. Sheep and cattle are preferred.

Endoparasitic infection or infestations suitable for treatment by the method of the invention include, for example and without limitation, liver flukes, tapeworms, strongyles, encysted Cyathostomes, pinworms, hairworms, whipworms, ascarids, large-mouth stomach worms, bots or the like.

In actual practice, the composition of the invention may be administered in dose rates of mg of active ingredient per kg of body weight of the host animal. Dose rates suitable for use in the method of invention will vary depending upon the mode of administration, the species and health of the host animal, the target parasite, the degree of infection or infestation, the breeding habitat, the potency of the macrocyclic lactone, and the like. In general, amounts of said composition sufficient to provide about 8.0 mg/kg to 15.0 mg/kg, preferably about 10 mg/kg to 12 mg/kg of praziquantel per body weight of the animal and about 0.5 mg/kg to 3.5 mg/kg, preferably about 1.0 mg/kg to 2.5 mg/kg of a macrocyclic lactone such as moxidectin per body weight of the animal and are suitable.

In certain preferred embodiments, when parenterally administered, the vaccine and pharmaceutical compositions of this invention are highly effective for protecting against or controlling helminthiasis, infection by acarids and arthropod endo- and ectoparasites and disease in warm-blooded animals such as sheep, cattle, horses, swine, deer, camels, poultry, dogs, cats and goats.

Immunogenic polypeptides may be derived from ticks such as species of Boophilus, Haemaphysalis, Otobius, Rhiphicephalus, Amblyomma, Dermacentor, Ixodes, and Hyaloma. More particularly, the polypeptide may be derived from Boophilus microplus, Boophilus (Rhipicephalus) annulatus, Boophilus decoloratus, Otobius megnini, Rhiphicephalus appendiculatus, Dermacentor andersoni, Dermacentor variabilis, Haemaphysalis longcomis, Amblyomma variegatum or Ixodes holocyclus.

Immunogenic polypeptides also may be derived from parasitic nematodes such as species of Strongylus, Trichostrongylus, Haemonchus, Ostertagia, Ascaris, Trichinella, Toxascaris, Uncinaria, Ancylostoma, Trichuris, Dirofilaria, Necator, Ancylostoma, Ascaris, Trichuris, Enterobius, Strongyloides, and Wuchereria. In particular, the at least one polypeptide may be derived from Strongylus vulgaris, Trichostrongylus colubriformis, Haemonchus contortus, Ostartagia ostertagi, Ascaris suum, Trichinella spiralis, Toxascaris leonine, Uncinaria stenocephala, Ancylostoma caninum, Trichuris vulpis, Dirofilaria immitis, Necator americanus, Ancylostoma duodenale, Ascaris lumbricoides, Trichuris trichiura, Enterobius vermicularus, Strongyloides stercoralis or Wuchereria bancrofti. The at least one polypeptide may also be derived from haematophageous parasitic insects, for example, species of Haemotobia, Hypoderma, Dermatobia, Anopheles, Lucilia, Ctenocephalides, Chrysomya, Gasterophulus, Culicoides, Stomoxys, Linognathus, Solenoptes, Haematopinus, Melophagus, Aedes, or Culex.

The at least one immunogenic polypeptide may also be derived from a haematophageous parasitic insect selected from the group consisting of Haemotobia spp, Hypoderma spp, Dermatobia spp, Anopheles spp, Lucilia spp, Ctenocephalides spp, Chrysomya spp, Gasterophulus spp, Culicoides spp, Stomoxys spp, Linognathus spp, Solenoptes spp, Haematopinus spp, Melophagus spp, Aedes spp, and Culex spp.

In another embodiment, the at least one immunogenic polypeptide is derived from a nematode selected from the group consisting of, Nematodirus spp, Dictyocaulus spp, Cooperia spp, Strongylus spp, Trichostrongylus spp, Haemonchus spp, Ostertagia spp, Ascaris spp, Trichinella spp, Toxascaris spp, Uncinaria spp, Ancylostoma spp, Trichuris spp, Dirofilaria spp, Necator spp, Ascaris spp, Trichuris spp, Enterobius spp, Strongyloides spp, and Wuchereria spp. In particular, the polypeptide may be derived from Strongylus vulgaris, Trichostrongylus colubriformis, Haemonchus contortus, Ostartagia ostertagi, Ascaris suum, Trichinella spiralis, Toxascaris leonina, Uncinaria stenocephala, Ancylostoma caninum, Trichuris vulpis, Dirofilaria immitis, Necator americanus, Ancylostoma duodenale, Ascaris lumbricoides, Trichuris trichiura, Enterobius vermicularus, Strongyloides stercorals and Wuchereria bancrofti.

In preferred embodiments, the at least one immunogenic polypeptide is derived from a trematode parasite (fluke) such as, which include, for example and without limitation, Fasciola hepatica, Fasciola gigantica, Fasciola magna, Schistosoma bovis, Schistosoma matthei, Schistosoma mansoni, Schistosoma haematobium, Schistosoma japonicum, Paramphistomum microbothium, Gigantocotyle explanatum, Dicrocoelium dendriticum, Eurytrema pancreaticum, Paragonimus westermani, Clonorchis sinensis and Opisthorchis viverrini.

More preferably, immunogenic polypeptides are derived from flukes such as Fasciola or Dicrocoelium, in particular from the liver fluke Fasciola hepatica. Molecules capable of stimulating an immune response which is effective against Fasciola or Dicrocoelium, in particular F. hepatica and F. gigantica, such are capable of conferring a cross-protective immune response.

Immunogenic Fasciola polypeptides for use in the compositions and methods described herein include, for example and without limitation, cathepsin proteases, e.g., cathepsin L or cathepsin L-like proteases and cathepsin B proteases, glutathione-S-transferase, dipeptidyl peptidase, excretory/secretory (E/S) products, peroxiredoxin, β-tubulin, α-tubulin, and haemoproteins isolatable from a species of Fasciola selected from F. hepatica, F. gigantica and F. magna and most preferably F. hepatica or a part, fragment or derivative thereof or as a fusion molecule comprising such a Fasciola polypeptide, a part, fragment or derivative thereof and which polypeptide is capable of stimulating antibody production in a suitable host against a Fasciola species. Highly preferred immunogenic polypeptides for use in the compositions and methods described herein are Fasciola cathepsin protease and Fasciola peroxiredoxin. Most preferred immunogenic polypeptides are cathepsin protease and peroxiredoxin derived from F. hepatica.

One of ordinary skill in the art will appreciate that such Fasciola cathepsin polypeptides and peroxiredoxin polypeptides may be present in a single, multivalent vaccine. In one embodiment, at least two different Fasciola cathepsin polypeptides are present in a single multivalent vaccine. For example, two different Fasciola hepatica cathepsin polypeptides, such as cathepsin L1 and cathepsin L3, may be present in a single multivalent vaccine. In another embodiment, a cathepsin B polypeptide can be combined with a cathepsin L polypeptide in a single multivalent vaccine. In a further embodiment, a cathepsin polypeptide can be combined with a peroxiredoxin polypeptide in a single multivalent vaccine. The invention is not limited by these examples.

Fasciola cathepsin antigen was described in International Patent Application No. WO94/09142 and U.S. Pat. No. 6,623,735. Immunogenic polypeptides may be derived from, for example and without limitation, Fasciola cathepsin polypeptides or fragments having the following accession numbers and sequences.

AAA29137
(SEQ ID NO: 1)
mrlviltllivgvfasnddlwhqwkriynkeykgadddhrrniweqnvkhiqehnlrhdlglvtyklglnqftdmtfeefkakylte
mprasellshgipykankravpdridwresgyvtevkdqggcgscwafsttgamegqymknektsisfseqqlvdcsgpfg
nygcngglmenayeylkrfgletessypyravegqcryneqlgvakvtgyytvhsgdevelqnlvgcrrpaavaldvesdfm
myrsgiyqsqtcspdrinhgvlavgygiqdgtdywivknswgtwwgedgyirmvrkrgnmcgiaslasvpmvaqfp
CAA80446
(SEQ ID NO: 2)
mrffvlavltvgvfasnddlwhqwkriynkeyngaddehrrniwgknvkhiqehnlrhglglvtyklglnqftdltfeefkakyliei
prssellsrgipykanklavpesidwrdyyyvtevkdqgqcgscwafsttgavegqfrknerasasfseqqlvdctrdfgnygc
gggymenayeylkhngletesyypyqavegpcqydgrlayakvtgyytvhsgdeielknlvgtedlpavaldadsdfmmy
qsgiyqsqtclpdrlthavlavgygsqdgtdywivknswgtwwgedgyirfarnrgnmcgiaslasvpmvarfp
CAA80450
(SEQ ID NO: 3)
qgqcgtcwafsttgtmegqymkkqrtsisfsdeqlvdcsrpwgnngcggglmenayqylkqfgletessypytavegqcry
neqlgvakvtgyytvhsgsevelknlvgsegparspvdvesdfmmyrsgiyqsqtclpfalnhavlavgygtqdgtdywivk
nsw
CAA80448
(SEQ ID NO: 4)
qgqcgtcwafattgvvegqysrkygsktgfseqqlvdcrrrhgnegcngglmtssyrylmnnslesegdypyeamdnrcra
nrtkgivkvksytvlknesethsrswsgtrgpvavgihaddgfqfyshgiyvsstcsswpanhgvlvvgygaeanspywivkn
tw
CAA80447
(SEQ ID NO: 5)
qgqcgrcwafsttgategqymknqrtsisfseqqlvdcsrdfgnygcngglmenayeylkrfgletessypyravegqcryne
qlgvakvtgyytvhsgdevelqnlvgagrpaavaldvesdfmmyrsgiyqsqtcspdrlnhgvlavgygtqdgtdywivknt
w
CAA80445
(SEQ ID NO: 6)
qgqcgwcwafsttgavegqfrknerasasfseqqlvdctrdfgnygcgggymenayeylkhngletesyypyqavegpcq
ydgrlayakvtgyytvhsgdeielknlvgtedlpavaldadsdfmmyqsgiyqsqtclpdrlthavlavgygsqdgtdywivkn
sw
CAA80444
(SEQ ID NO: 7)
qgqcgwcwafsttgalegqymksqrinisfseqqlvdcsgdfgnhgcsgglmekayeylrhfgletessysyradegpcqyd
rqlgvaqvsgyfivhsqdevalknligvegpaavaldvnidfmmyrsgiyqdeicssrylnhavlavgygtedgtdywivkntw
Q24940
(SEQ ID NO: 8)
mrlfilavltvgvlgsnddlwhqwkrmynkeyngaddqhrrniweknvkhiqehnlrhdlglvtytlglnqftdmtfeefkakylte
msrasdilshgvpyeannravpdkidwresgyvtevkdqgncgscwafsttgtmegqymknertsisfseqqlvdcsgpw
gnngcsgglmenayqylkqfgletessypytavegqcrynkqlgvakvtgyytvhsgsevelknlvgarrpaavavdvesdf
mmyrsgiyqsqtcsplrvnhavlavgygtqggtdywivknswgtywgergyirmarnrgnmcgiaslaslpmvarfp
P80528
(SEQ ID NO: 9)
sndvswhewkrmynkeynga
CAC12806
(SEQ ID NO: 10)
snddlwhqwkrmynkeyngaddehrrniweenvkhiqehnlrhdlglvtytlglnqftdmtfeefkakyltemprasdilshgi
pyeannravpdkidwresgyvtgvkdqgncgscwafsttgtmegqymknektsisfseqqlvdcsgpwgnngcsgglme
nayeylkrfgletessypyravegqcryneqlgvakvtgyytvhsgsevelknlvgsegpaaiaveaesdfmmyrsgiyqsqt
clpfalnhavlavgygtqdgtdywivknswglswgergyirmarnrgnmcgiaslaslpmvarfp
AAR99518
(SEQ ID NO: 11)
mrlfilavltvgvlgsnddlwhqwkrmynkeyngaddehrrniweenvkhiqehnlrhdlglvtytlglnqftdmtfeefkakylt
emsrasdilshgvpyetnnravpdkidwresgyvtevkdqgncgscwafsttgtmegqymknertsisfseqqlvdcsgpw
gnngcsgglmenayqylkqfgletessypytavegqcryneqlgvakvtgyytvhsgsevelknlvgsegpaavavdvesd
fmmyrsgiyqsqtcsplsvnhavlavgygtqggtdywivknswglswgergyirmyrnrgnmcgiaslaslpmvarfp
AAR99519
(SEQ ID NO: 12)
msrasdilshgipyeannravpdkidwresgyvtgvkdqgncgscwafsttgtmegqymknertsisfseqqlvdcsgpwg
nngcsgglmenayqylkqfgletessypytavegqcrynrqlgvakvtgyytvhsgsevelknlvgsegpaaiavdvesdfm
myrsgiyqsqtclpfalnhavlavgygtqggtdywivknswglswgergyirmarnrgnmcgiaslaslpmvarfp
Q09093
(SEQ ID NO: 13)
avpdkidpresgyvtgvkdq

Immunogenic polypeptides can also be derived from, for example and without limitation, Fasciola cathepsin polypeptides or fragments having the following accession numbers and SEQ ID Nos: AAA29137 (SEQ ID NO: 14); ABG00259 (SEQ ID NO: 15); ABZ80398 (SEQ ID NO: 16); ABF85681 (SEQ ID NO: 17); ABZ80399 (SEQ ID NO: 18); CAC12805 (SEQ ID NO: 19); CAC12807 (SEQ ID NO: 20); ABF85682 (SEQ ID NO: 21), ABZ80401 (SEQ ID NO: 22); ABZ80400 (SEQ ID NO: 23); AAF76330 (SEQ ID NO: 24); ABZ80402 (SEQ ID NO: 25); ABU62925 (SEQ ID NO: 26); AAD11445 (SEQ ID NO: 27); CAO98753 (SEQ ID NO: 28); CAD32937 (SEQ ID NO: 29); ABF85680 (SEQ ID NO: 30); ABF85679 (SEQ ID NO: 31); and ABF85678 (SEQ ID NO: 32).

Fasciola dipeptidyl peptidase antigen was described in more detail in International Patent Application No. WO94/28925 and U.S. Pat. No 5,885,814.

Fasciola hemoprotein molecules were described in International Patent Application No. PCT/GB95/02350. Examples Fasciola hemoprotein polypeptides are available, for example and without limitation, under accession numbers ABW96608 (hemoglobin) and NP 066225, NP 066225, AAG13153, AAG13154, NP 006217, AAG13145, AAB01231, CAF31342, CAF31343, CAF31344, CAF31345, CAF31346 and CAF31347 (cytochrome c oxidase).

Examples Fasciola glutathione-S-transferase polypeptides are available, for example and without limitation, under accession numbers AAB28746, 2FHE B, 2FHE A, P31670, P56598, P30112, P31671, 1FHE A, 1905266D, CAA00118, CAA00119, CAA00120, CAA00121, CAA00122.

Examples of Fasciola α-tubulin polypeptides are available, for example and without limitation, accession numbers CAO79602, CAO79603, CAO79604, CAO79605, CAO79606, CAP72044, CAP72045, CAP72046, CAP72047, and CAP72048.

Fasciola peroxiredoxin and β-tubulin molecules were described in U.S. Pat. No. 6,676,944. Examples of Fasciola β-tubulin polypeptides are available, for example and without limitation, accession numbers CAC82577, CAO79607, CAO79608, CAO79609, CAO79610, CAO79611, CAO79612, CAP72049, CAP72050, CAP72051, CAP72052, CAP72053 and CAP72054.

Immunogenic polypeptides may be derived from, for example and without limitation, Fasciola peroxiredoxin polypeptides or fragments having the following accession numbers and sequences.

AAB71727
(SEQ ID NO: 33)
mlqpnmpapnfsgqavvgkefetislsdykgkwvilafypldftfvcpteiiaisdqmeqfaqrncavifcstdsvyshlqwtkm
drkvggigqlnfplladknmsvsrafgvldeeqgntyrgnflidpkgvlrqitynddpvgrsveealrlldafifheehgevcpan
wkpksktivptpdgskayfssan
P91883
(SEQ ID NO: 34)
mlqpnmpapnfsgqavvgkefetislsdykgkwvilafypldftfvcpteiiaisdqmeqfaqrncavifcstdsvyshlqwtkm
drkvggigqlnfplladknmsvsrafgvldeeqgntyrgnflidpkgvlrqitvnddpvgrsveealrlldafifheehgevcpan
wkpksktivptpdgskayfssan
ACI04165
(SEQ ID NO: 35)
mcdrdqcspgrhplphshphlqrpmlqpnmpapnfsgqavvgkefktislsdykgkwvilafypldftfvcpteiiafsdqme
qfarrncavifcstdsvyshlqwtkmdrkvggigqlnfplladknmsisraygvldeeqgntyrgnflidpkgvlrqitvndrpvgr
sveealrlldafifheehgevcpanwkpksktivptpdgskayfssan
CAA06158
(SEQ ID NO: 36)
mlqpnmpapnfsgqavvgkefktislsdykgkwvilafypldftfvcpteiiafsdqmeqfarrncavifcstdsvyshlqwtkm
drkvggigqlnfpllaeknmsisraygvldeeqgntyrgnflidpkgvlpqitvndrpvgrsveealrlldafifheehgevcpanw
kpksktivptpdgskayfssan

Fasciola immunogenic polypeptides may be isolated from immature (e.g., newly excysted larval stage) or mature Fasciola species although the mature organism is the preferred source. More preferably, Fasciola immunogenic polypeptides are prepared recombinantly and purified using methods well known in the art.

In a preferred embodiment, the invention provides the invention provides compositions and methods for the protection and or treatment of fascioliasis, which is caused by infection with the trematode parasite Fasciola hepatica. Accordingly, the invention provides compositions and methods for the protection and or treatment of fascioliasis in a ruminant animal, such as for example and without limitation, sheep and cattle. Species of Fasciola include but are not limited to F. hepatica, F. gigantica or F. magna. The most preferred species is F. hepatica. The Fasciola species may be in the mature state or in the newly excysted larval stage trematode parasite, Fasciola hepatica.

At least one dosage unit per animal is contemplated herein as a vaccination regimen. In some embodiments, two or more dosage units may be especially useful. A dosage unit may typically be about 0.1 to 10 milliliters of vaccine composition, preferably about 0.5 to 5 milliliters, and even more preferably about 1 to 2 milliliters, with each dosage unit containing the heretofore described quantity of antigen component. The skilled artisan will quickly recognize that a particular quantity of vaccine composition per dosage unit, as well as the total number of dosage units per vaccination regimen, may be optimized, so long as an effective immunizing amount of the virus or a component thereof is ultimately delivered to the animal.

Vaccine compositions may be administered parenterally, for example, intramuscularly, subcutaneously, intraperitoneally, intradermally or the like, preferably intramuscularly; or said composition may be administered orally or intranasally. Routes of administration of vaccine formulations, dosages to be administered, and frequency of injections are factors that can be optimized using ordinary skill in the art. Preferably, a vaccine composition is administered parenterally, more preferably by subcutaneous injection. Typically, an initial vaccination is followed some weeks later by one or more “booster” vaccinations, the net effect of which is the production of vigorous cellular and humoral immune response against the immunogenic polypeptide.

In actual practice, vaccine compositions are administered parenterally, subcutaneously, orally, intranasally, or by other available means, preferably parenterally, more preferably intramuscularly, in effective amounts according to a schedule which may be determined by the time of anticipated potential exposure to pathogenic agents or carriers. In this way, the treated animal will have time to build immunity prior to the natural exposure. By way of a non-limiting example, a typical treatment schedule or dosing regimen may include parenteral administration, preferably intramuscular injection of one dosage unit, at least about 2-8 weeks prior to potential exposure. At least two administrations are preferred, for example one dosage unit at about 8 weeks and a second dosage unit at about 3-5 weeks prior to potential exposure of the treated animal. As heretofore set forth, a dosage unit will typically be within the range of about 0.1 to 10 milliliters of a vaccine composition containing the previously described amounts of active and percentages of adjuvant, immunogenic polypeptide and inactive(s) and further comprising a macrolide compound, as set forth above. A dosage unit within the range of about 0.5 to 5 milliliters is perhaps more preferred, with about 1 to 2 milliliter(s) being particularly preferred.

In order to facilitate a further understanding of the invention, the following examples are presented primarily for the purpose of illustrating more specific details thereof. The invention is not to be deemed limited thereby except as defined in the claims.

### EXAMPLES

### Example 1

### Adjuvant Compositions (AC)

Adjuvant compositions (ACs) were formulated by combining the following ingredients.

| | |
|---|---|
| Component | Amount (ml) |

Adjuvant Composition 1 (AC 1): SP Oil/SLCD

| | |
|---|---|
| 0.01M PBS | 0.7 |
| SLCD (sulfolipo-cyclodextrin) adjuvant | 0.1 |
| SP Oil with thimerosal | 0.2 |
| | 1.0 Total |

Adjuvant Composition 2 (AC 2): Montanide

| | |
|---|---|
| 0.01M PBS | 0.5 |
| Montanide ISA 206 VG | 0.5 |
| | 1.0 Total |

Adjuvant Composition 3 (AC 3): SP Oil/SLCD + BSA

| | |
|---|---|
| 0.01M PBS | 0.6 |
| BSA (1.0 mg/ml) | 0.1 |
| SLCD adjuvant | 0.1 |
| SP Oil with thimerosal | 0.2 |
| | 1.0 Total |

Adjuvant Composition 4 (AC 4): Montanide + BSA

| | |
|---|---|
| 0.01M PBS | 0.4 |
| BSA (1.0 mg/ml) | 0.1 |
| Montanide ISA 206 VG | 0.5 |
| | 1.0 Total |

Each AC had a milky appearance, indicating the presence of an emulsion.

### Example 2

### Moxidectin/Adiuvant Formulations

The following PBS (0.01 M) control and moxidectin/adjuvant compositions were formulated at room temperature.

| | Formulation | | | | |
|---|---|---|---|---|---|
| | PBS control | 1 | 2 | 3 | 4 |
| Component | % w/v | % w/v | % w/v | % w/v | % w/v |
| Moxidectin | 1.0 | 1.0 | 1.0 | 1.0 | 1.0 |
| Benzyl | 4.0 | 4.0 | 4.0 | 4.0 | 4.0 |
| alcohol | | | | | |
| BHT | 0.3 | 0.3 | 0.3 | 0.3 | 0.3 |
| Polysorbate | 20.0 | 20.0 | 20.0 | 20.0 | 20.0 |
| 80 | | | | | |
| Propylene | 20.0 | 20.0 | 20.0 | 20.0 | 20.0 |
| glycol | | | | | |
| EDTA- | 0.027 | 0.027 | 0.027 | 0.027 | 0.027 |
| disodium, | | | | | |
| dihydrate | | | | | |
| PBS Control | q.s. to | — | — | — | — |
| | 100 ml | | | | |
| AC 1 | — | q.s. to | — | — | — |
| (SP | | 100 ml | | | |
| Oil/SLCD) | | | | | |
| AC 2 | — | — | q.s. to | — | — |
| (Montanide | | | 100 ml | | |
| ISA 206 VG) | | | | | |
| AC 3 | — | — | — | q.s. to | — |
| (BSA w/SP | | | | 100 ml | |
| Oil and | | | | | |
| SLCD) | | | | | |
| AC 4 | — | — | — | — | q.s. to |
| (BSA | | | | | 100 ml |
| w/montanide | | | | | |
| ISA 206 VG) | | | | | |

Procedure

A moxidectin solution was formed by stirring moxidectin in benzyl alcohol until the moxidectin was dissolved and then adding BHT with continued stirring until the BHT was dissolved. The resulting moxidectin solution was a clear yellow solution. Moxidectin solution was stored in the refrigerator prior to using in formulations. For the Control PBS formulation, PBS (13.0 g) was mixed with 7.2 mg of EDTA until the EDTA had dissolved and 8.42 g of the resulting solution was transferred to a 25 ml volumetric flask. Separately, polysorbate 80 was added to moxidectin/BHT solution, followed by addition of propylene glycol. The resulting mixture of moxidectin/benzyl alcohol/BHT/polysorbate 80/propylene glycol was stirred until homogeneous and then transferred to the volumetric flask. The flask was rinsed with the remainder of the PBS/EDTA solution and transferred to the volumetric flask. Additional PBS was added to the flask to a final volume of 25 ml and the flask was inverted to achieve good mixing and homogeneity.

For moxidectin/adjuvant formulations 1-4, adjuvant compositions 1-4 (AC 1-4; 12.0 g, respectively) were added to 7.2 mg of EDTA and mixed to dissolve the EDTA. The resulting compositions were transferred to a 25 ml volumetric flask. Separately, moxidectin/BHT solution, polysorbate 80 and polypropylene glycol were mixed until homogeneous. The resulting mixture of moxidectin/benzyl alcohol/BHT/polysorbate 80/propylene glycol retained the clear, yellow appearance of the moxidectin/BHT solution. Moxidectin/benzyl alcohol/BHT/polysorbate 80/propylene glycol mixture (11.3 g) was transferred to the volumetric flask and mixed by swirling and the volume of flask was brought to 25 ml with adjuvant composition and the flask was inverted to achieve good mixing and homogeneity.

### Example 3

### The Emulsion Properties and Stability of Formulations containing Moxidectin

Aliquots of the moxidectin/adjuvant formulations from Example 2 were placed small borosilicate clear vials, stopped with Teflon coated stoppers, and sealed. The formulations were observed for their appearances at 4° C., 25° C./65% relative humidity (RH), and 40° C./20% RH over time. The results for the appearance and the presence of phase separation for the formulation over time was as follows:

| | Time/Condition | | | | |
|---|---|---|---|---|---|
| | Immediately | Refrigerated for | | | 1 month at 40° C. |
| | upon Formation | 2 hrs immediately | 1 month | 1 month | then temperature |
| Formulation | at Room Temp | following formulation | at 4° C. | at 40° C. | brought to Room Temp |
| PBS Control | Clear | Clear | Clear | clear | Clear |
| 1 | Slightly Hazy | Very hazy* | Slightly | Clear | Slightly |
| Moxidectin/ | | | hazy | | hazy - clear |
| SP Oil/SLCD | | | | | |
| 2 | Milky | Milky** | Phase | Clear | Phase |
| Moxidectin/ | | | separation | | separate |
| montanide | | | (milky on top, | | |
| | | | clear on bottom) | | |
| 3 | Slightly Hazy | Very hazy* | Slightly | Clear | Slightly |
| Moxidectin/BSA/ | | | hazy | | hazy - clear |
| SP Oil/SLCD | | | | | |
| 4 | Milky | Milky** | Phase | Clear | Phase |
| Moxidectin/BSA/ | | | separation | | separate |
| montanide | | | | | |

*indicative of a true emulsion (oil/water) when haziness increases as temperature decreases. No precipitation or phase separation was observed after 1 month at 4° C., 25° C., and 40° C.
**phase separation under all storage conditions

The results indicated that the initially moxidectin/adjuvant formulations, both with and without protein, formed emulsions. The formulations of Moxidectin/SP Oil/SLCD remained as emulsions following one month at 4° C.

### Example 4

### Moxidectin Stability in the Vaccine Formulations

The stability of moxidectin in the Moxidectin/Adjuvant Formulations was determined by using HPLC to determine the amount of moxidectin in the moxidectin/emulsion Formulations 1-4. No significant reduction in the amount of moxidectin present was observed for any of Formulations 1-4 after 1 month at either 4° C. or 40° C. Moxidectin is thus stable in the formulations for at least 1 month.

What is claimed:

1. A composition comprising an oil adjuvant, a macrocyclic lactone effective for the prevention or control of parasitic infection in a warm-blooded animal and an immunizing amount of at least one immunogenic polypeptide.

2. The composition of claim 1 wherein said macrocylic lactone is effective for protecting or controlling helminthiasis in said warm blooded animal.

3. The composition of claim 1 wherein said macrocylic lactone is a milbemycin compound, an avermectin or a combination thereof.

4. The composition of claim 3 wherein said milbemycin compound is selected from moxidectin, nemadectin, milbemycin, milbemycin oxime or combinations thereof.

5. The composition of claim 3 wherein said avermectin compound is selected from abamectin, doramectin, ivermectin, selamectin, emamectin, eprinomectin, or combinations thereof.

6. The composition of claims 1 comprising said macrocylic lactone in the range of about 0.01 to about 2.0%, about 0.1 to about 1.0% w/v, 0.1% to about 10.0, about 0.5% to about 5%, about 0.5% to about 2%, about 0.5 to about 3.0%, or about 1.0 to about 2.5% of said composition, on a weight/volume (w/v) basis.

7. The composition of claims 1 wherein said oil adjuvant comprises a non-metabolizable mineral oil, a metabolizable oil, or a combination thereof.

8. The composition of claim 7 wherein said oil adjuvant comprises a metabolizable oil.

9. The composition of claim 8 wherein said oil adjuvant is an oil-in-water emulsion.

10. The composition of claim 7 wherein said oil adjuvant comprises, SP Oil, Emulsigen, Montanide™, sulfolipo-cyclodextrin in squalane in water emulsion (SL-CD) or combinations thereof.

11. The composition of claim 10 wherein said oil adjuvant comprises SP Oil.

12. The composition of claim 1 comprising said oil adjuvant in the range of about 1% to about 50%, about 5% to about 50%, or about 15% to about 30% of said composition on a volume/volume (v/v) basis.

13. The composition of claim 1 further comprising a dispersing agent.

14. The composition of claim 13 wherein said dispersing agent is selected from a polyethylene oxide sorbitan mono-oleate, polyoxyethylene alcohol, polyethylene glycols, α-hydro-ω-hydroxypoly(oxyethylene) poly(oxypropylene)poly(oxyethylene)block copolymer, or a combination thereof.

15. The composition of claim 1 comprising one or more water soluble organic solvent.

16. The composition of claim 15 wherein said one or more water soluble organic solvent is selected from benzyl alcohol, propylene glycol, or a combination thereof.

17. The composition of claim 15 comprising said one or more water soluble organic solvent in a range of about 1% to about 40%, about 5% to about 40%, about 5% to about 30%, about 5% to about 25% or about 10% to about 25% of said composition, on a v/v basis.

18. The composition of claim 1 wherein said at least one immunogenic polypeptide is derived from a nematode or trematode.

19. The composition of claim 18 wherein said at least one immunogenic polypeptide is derived from a Fasciola species polypeptide.

20. The composition of claim 19 wherein said at least one immunogenic polypeptide is derived from a cathepsin protease, glutathione-S-transferase, dipeptidyl peptidase, excretory/secretory (E/S) product, peroxiredoxin, β-tubulin, α-tubulin, or haemoprotein.

21. The composition of claim 20 wherein said at least one immunogenic polypeptide is derived from a cathepsin protease, peroxiredoxin, or a combination thereof.

22. The composition of claim 21 wherein said at least one immunogenic polypeptide is Fasciola hepatica cathepsin protease or peroxiredoxin, a fragment of at least 8 amino acids thereof or a polypeptide comprising a sequence having at least 80% or at least 90% identity when compared to said cathepsin protease, peroxiredoxin, or fragment of at least 8 amino acids thereof.

23. The composition of claim 22 wherein said cathepsin protease has the sequence set forth in SEQ ID NO: 1-32.

24. The composition of claim 22 wherein said peroxiredoxin has the sequence set forth in SEQ ID NO: 33-36.

25. The composition of claim 1 in the form of an oil-in-water emulsion.

26. The composition of claim 25 wherein said emulsion comprises particles having an average size of less than or equal to 500 μm.

27. A method for the prevention or control of a parasitic infection in a warm blooded mammal, comprising administering an effective amount of the composition of claim 1 to said warm blooded mammal, to prevent or control said parasitic infection.

28. The method of claim 27 for the prevention or control of helminthiasis.

29. The method of claim 27 wherein said warm blooded animal is selected from dog, cat, cattle, sheep, swine, horse, goat or avian species.

30. The method of claim 27 for the prevention or control of infection by a Fasciola species.


SEQUENCE LISTING (36 sequences)

SEQ ID NO: 1 - protein, 326 aa, Fasciola hepatica
  MISC_FEATURE (1)..(326): cathepsin L-like protease
MRLVILTLLIVGVFASNDDLWHQWKRIYNKEYKGADDDHRRNIWEQNVKHIQEHNLRHDLGLVTYKLGLNQFTDMTFEEFKAKYLTEMPRASELLSHGIPYKANKRAVPDRIDWRESGYVTEVKDQGGCGSCWAFSTTGAMEGQYMKNEKTSISFSEQQLVDCSGPFGNYGCNGGLMENAYEYLKRFGLETESSYPYRAVEGQCRYNEQLGVAKVTGYYTVHSGDEVELQNLVGCRRPAAVALDVESDFMMYRSGIYQSQTCSPDRLNHGVLAVGYGIQDGTDYWIVKNSWGTWWGEDGYIRMVRKRGNMCGIASLASVPMVAQFP

SEQ ID NO: 2 - protein, 326 aa, Fasciola hepatica
  MISC_FEATURE (1)..(326): cathepsin L-like protease
MRFFVLAVLTVGVFASNDDLWHQWKRIYNKEYNGADDEHRRNIWGKNVKHIQEHNLRHGLGLVTYKLGLNQFTDLTFEEFKAKYLIEIPRSSELLSRGIPYKANKLAVPESIDWRDYYYVTEVKDQGQCGSCWAFSTTGAVEGQFRKNERASASFSEQQLVDCTRDFGNYGCGGGYMENAYEYLKHNGLETESYYPYQAVEGPCQYDGRLAYAKVTGYYTVHSGDEIELKNLVGTEDLPAVALDADSDFMMYQSGIYQSQTCLPDRLTHAVLAVGYGSQDGTDYWIVKNSWGTWWGEDGYIRFARNRGNMCGIASLASVPMVARFP

SEQ ID NO: 3 - protein, 166 aa, Fasciola hepatica
  MISC_FEATURE (1)..(166): cathepsin L-like protease
QGQCGTCWAFSTTGTMEGQYMKKQRTSISFSDEQLVDCSRPWGNNGCGGGLMENAYQYLKQFGLETESSYPYTAVEGQCRYNEQLGVAKVTGYYTVHSGSEVELKNLVGSEGPARSPVDVESDFMMYRSGIYQSQTCLPFALNHAVLAVGYGTQDGTDYWIVKNSW

SEQ ID NO: 4 - protein, 166 aa, Fasciola hepatica
  MISC_FEATURE (1)..(166): cathepsin L-like protease
QGQCGTCWAFATTGVVEGQYSRKYGSKTGFSEQQLVDCRRRHGNEGCNGGLMTSSYRYLMNNSLESEGDYPYEAMDNRCRANRTKGIVKVKSYTVLKNESETHSRSWSGTRGPVAVGIHADDGFQFYSHGIYVSSTCSSWPANHGVLVVGYGAEANSPYWIVKNTW

SEQ ID NO: 5 - protein, 166 aa, Fasciola hepatica
  MISC_FEATURE (1)..(166): cathepsin L-like protease
QGQCGRCWAFSTTGATEGQYMKNQRTSISFSEQQLVDCSRDFGNYGCNGGLMENAYEYLKRFGLETESSYPYRAVEGQCRYNEQLGVAKVTGYYTVHSGDEVELQNLVGAGRPAAVALDVESDFMMYRSGIYQSQTCSPDRLNHGVLAVGYGTQDGTDYWIVKNTW

SEQ ID NO: 6 - protein, 166 aa, Fasciola hepatica
  MISC_FEATURE (1)..(166): cathepsin L-like protease
QGQCGWCWAFSTTGAVEGQFRKNERASASFSEQQLVDCTRDFGNYGCGGGYMENAYEYLKHNGLETESYYPYQAVEGPCQYDGRLAYAKVTGYYTVHSGDEIELKNLVGTEDLPAVALDADSDFMMYQSGIYQSQTCLPDRLTHAVLAVGYGSQDGTDYWIVKNSW

SEQ ID NO: 7 - protein, 166 aa, Fasciola hepatica
  MISC_FEATURE (1)..(166): cathepsin L-like protease
QGQCGWCWAFSTTGALEGQYMKSQRINISFSEQQLVDCSGDFGNHGCSGGLMEKAYEYLRHFGLETESSYSYRADEGPCQYDRQLGVAQVSGYFIVHSQDEVALKNLIGVEGPAAVALDVNIDFMMYRSGIYQDEICSSRYLNHAVLAVGYGTEDGTDYWIVKNTW

SEQ ID NO: 8 - protein, 326 aa, Fasciola hepatica
  MISC_FEATURE (1)..(326): cathepsin L-like protease
MRLFILAVLTVGVLGSNDDLWHQWKRMYNKEYNGADDQHRRNIWEKNVKHIQEHNLRHDLGLVTYTLGLNQFTDMTFEEFKAKYLTEMSRASDILSHGVPYEANNRAVPDKIDWRESGYVTEVKDQGNCGSCWAFSTTGTMEGQYMKNERTSISFSEQQLVDCSGPWGNNGCSGGLMENAYQYLKQFGLETESSYPYTAVEGQCRYNKQLGVAKVTGYYTVHSGSEVELKNLVGARRPAAVAVDVESDFMMYRSGIYQSQTCSPLRVNHAVLAVGYGTQGGTDYWIVKNSWGTYWGERGYIRMARNRGNMCGIASLASLPMVARFP

SEQ ID NO: 9 - protein, 20 aa, Fasciola hepatica
  MISC_FEATURE (1)..(20): cathepsin L-like protease
SNDVSWHEWKRMYNKEYNGA

SEQ ID NO: 10 - protein, 311 aa, Fasciola hepatica
  MISC_FEATURE (1)..(311): cathepsin L1
SNDDLWHQWKRMYNKEYNGADDEHRRNIWEENVKHIQEHNLRHDLGLVTYTLGLNQFTDMTFEEFKAKYLTEMPRASDILSHGIPYEANNRAVPDKIDWRESGYVTGVKDQGNCGSCWAFSTTGTMEGQYMKNEKTSISFSEQQLVDCSGPWGNNGCSGGLMENAYEYLKRFGLETESSYPYRAVEGQCRYNEQLGVAKVTGYYTVHSGSEVELKNLVGSEGPAAIAVEAESDFMMYRSGIYQSQTCLPFALNHAVLAVGYGTQDGTDYWIVKNSWGLSWGERGYIRMARNRGNMCGIASLASLPMVARFP

SEQ ID NO: 11 - protein, 326 aa, Fasciola hepatica
  MISC_FEATURE (1)..(326): cathepsin L protein
MRLFILAVLTVGVLGSNDDLWHQWKRMYNKEYNGADDEHRRNIWEENVKHIQEHNLRHDLGLVTYTLGLNQFTDMTFEEFKAKYLTEMSRASDILSHGVPYETNNRAVPDKIDWRESGYVTEVKDQGNCGSCWAFSTTGTMEGQYMKNERTSISFSEQQLVDCSGPWGNNGCSGGLMENAYQYLKQFGLETESSYPYTAVEGQCRYNEQLGVAKVTGYYTVHSGSEVELKNLVGSEGPAAVAVDVESDFMMYRSGIYQSQTCSPLSVNHAVLAVGYGTQGGTDYWIVKNSWGLSWGERGYIRMVRNRGNMCGIASLASLPMVARFP

SEQ ID NO: 12 - protein, 239 aa, Fasciola hepatica
  MISC_FEATURE (1)..(239): cathepsin L protein
MSRASDILSHGIPYEANNRAVPDKIDWRESGYVTGVKDQGNCGSCWAFSTTGTMEGQYMKNERTSISFSEQQLVDCSGPWGNNGCSGGLMENAYQYLKQFGLETESSYPYTAVEGQCRYNRQLGVAKVTGYYTVHSGSEVELKNLVGSEGPAAIAVDVESDFMMYRSGIYQSQTCLPFALNHAVLAVGYGTQGGTDYWIVKNSWGLSWGERGYIRMARNRGNMCGIASLASLPMVARFP

SEQ ID NO: 13 - protein, 20 aa, Fasciola hepatica
  MISC_FEATURE (1)..(20): cathepsin L protein
AVPDKIDPRESGYVTGVKDQ

SEQ ID NO: 14 - protein, 326 aa, Fasciola hepatica; identical to SEQ ID NO: 1
  MISC_FEATURE (1)..(326): cathepsin L2

SEQ ID NO: 15 - protein, 219 aa, Fasciola hepatica
  MISC_FEATURE (1)..(219): cathepsin L2
RIRLCDWRDYYYVTEVKDQGQCGSCWAFSTTGAVEGQFRKNERASASFSEQQLVDCTRDFGNYGCGGGYMENAYEYLKHNGLETESYYPYQAVEGPCQYDGRLAYAKVTGYYTVHSGDEIELKNLVGTEGPAAIAVDVESDFMMYRSGIYQSQTCLPFALNHAVLAVGYGTQDGTDYWIVKNSWGLSWGERGYIRMARNRGNMCGIASLASLPMVARFP

SEQ ID NO: 16 - protein, 306 aa, Fasciola hepatica
  MISC_FEATURE (1)..(306): cathepsin L3
WHQWKRMYNKEYNGADDEHRRNIWEKNVKHIEEHNLRHDRGLVTYKLGLNQFTDLTFEEFKAKYLMEMSLVSESLSDGISYEAEGNDVPASVDWREYGYVTEVKDQGQCGSCWAFSAVGAIEGQYLRKFQNQTLFSEQQLVDCTRRFGNHGCGGGWMENAYKYLKNSGLETASDYPYQGWEYQCQYRKELGVAKVTGAYTVHSGDEMKLMQMVGREGPAAVAVDAQSDFYMYESGIFQSQTCTSRSVTHAVLAVGYGTESGTDYWILKNSWGKWWGEDGYMRFARNRNNMCAIASVASVPMVERFP

SEQ ID NO: 17 - protein, 226 aa, Fasciola hepatica
  MISC_FEATURE (1)..(226): cathepsin L3
WKRMYNKEYNGADDEHRRNIWEQNVKHIEEHNLRHDRGLVTYKLGLNQFTDLTFEEFKAKYLMEMSPESESLSDGISYEAEGNDVPASIDWRQYGYVTEVKDQGQCGSCWAFSAVGAIEGQYLKKFQNQTLFSEQQLVDCTRRFGNHGCGGGWMENAYKYLKNSGLETASYYPYQGWEYQCQYRKELGVAKVTGAYTVHSGDEMKLMQMVGREGPAAVAVDAQPDF

SEQ ID NO: 18 - protein, 306 aa, Fasciola hepatica
  MISC_FEATURE (1)..(306): cathepsin L3
WHQWKRIYNKEYNGADNEHRRKIWEQNVKHIQEHNLRHDLGLVTYTLGLNQFTDLTFEEFKAKYLIEMSLESESLSDGISYEAEGNDVPASIDWRQYGYVTEVKDQGQCGSCWAFSAVGAIEGQYVKKFQNQTLFSEQQLVDRTRRFGNHGCGGGWMENAYKYLKNSGLETASYYPYQAWEYPCQYRRELGVAKVTGAYTVHSGDEMRLMQMVGREGPAAVAVDAQSDFYMYKSGIFQSQTCTSQRVTHPVLAVGYGTESGTDYWILKNRWGKWWGEDGYMRFARNRNNMCAIASVASVPMVERFP

SEQ ID NO: 19 - protein, 311 aa, Fasciola hepatica
  MISC_FEATURE (1)..(311): procathepsin L3
SNDVSWHEWKRMYNKEYNGADEEHRRNIWGKNVKHIEEHNLRHDRGLVTYKLGLNQFTDPTFEEFQAKYLMEMSPVSESLSDGVSYEAEGNDVPASIDWREYGYVTEVKDQGQCGSCWAFSAVGAIEGQYVKKFQNQTLFSEQQLVDCTRRFGNHGCGGGWMENAYKYLKNSGLETASYYPYQAVEYQCQYRKELGVAKVTGAYTVHSGDEMKLMPMVGREGPAAVAVDAQSDFYMYESGIFQSQTCTSRSVTHAVLAVGYGTESGTDYWILKNSWGKWWGEDGYMRFARNRGNMCAIASVASVPMVERFP

SEQ ID NO: 20 - protein, 306 aa, Fasciola hepatica
  MISC_FEATURE (1)..(306): procathepsin L3
WHEWKRMYNKEYNGADDEHRRNIWEQNAKHIEEHNLRHDRGLVTYKLGLNQFTDLTFEEFKAKYLMEMSPVSESLSDGISYEAEGKDVPASIDWRQYGYVTEVKDQGQCGSCWAFSPVGAIEGQYVKKFQNQTLFSEQQLVDCTRRFGNHGCGGGWMENAYKYLKNSGLETASDYPYQGWEYQCQYRKELGVAKVTGAYTVHSGDEMKLMPMVRKKGPAAAAVDAQPDFYMYESGIFQSQYCSSRRVTHAVLAVGHGTESGTDYWILKNSWGKWWGEDGYMRFARNRGNMCAIASVASVPMVERFP

SEQ ID NO: 21 - protein, 226 aa, Fasciola hepatica
  MISC_FEATURE (1)..(226): cathepsin L4
WKRMYNKEYNGVDDVHRRNIWEENVKHIQEHYIRHDLGLVTYTLGLNQFTDMTFEEFKAKYLREIPRASDMLSHGIPYEANDRAVPESIDWREFGYVTEVKDQGDCGSCWAFSTTGAVEGQYMKNPKANISFSEQQLVDCSGDYGNHGCNGGFMENAYEYLERRGLETESSYPYKAEEGPCKYDSRLGVAKVNGYYIDHSGIESKLAHLVGDEGPAAVAVDAQPDF

SEQ ID NO: 22 - protein, 303 aa, Fasciola hepatica
  MISC_FEATURE (1)..(303): cathepsin L4
WKRMYNKEYNGVDDVHRRNIWEENVKHIQEHNIRHDLGLVTYTLGLNQFTDMTFEEFKAKHLREIPRASDMLSHGIPYEANDRAVPESIDWREFGYVTEVKDQGDCGSCWAFSTTGAVEGQYMKNPKANISFSEQQLVDCSGDYGNHGCNGGFMENAYEYLERRGLETESSYPYKAEEGPCKYDSRLGVVEVFGYFIEHSGIESKLAHLVGDKGPAAVAVDVESDFLMYRGGIYASRNCSSEKLNHAMLVVGYGTQDGTDYWIVKNSWGSLWGDHGYIRMARNRDNMCGIASAASVPVVEGFL

SEQ ID NO: 23 - protein, 303 aa, Fasciola hepatica
  MISC_FEATURE (1)..(303): cathepsin L4
WKRMCNKEYKGVDDVHRRNIWEENVKHIQEHNIRHDLGLVTYTLGLSQFTDMTFEEFKATYLREIPRASDMLSHGIPYEANDRAVPESIDWREFGYVTEVKDQGDCGSCWAFSTTGAVEGQYTKNQKANISFSEQQLVDCSGDYGNHGCNGGFMENAYEYLERRGLETESSYPYKAEEGPCKYDSRLGVVEVFGYFIEHSGIESKLAHLVGDKGPAAVAVDVESDFLMYRGGIYASRNCSSESLNHGILVVGYGTQDGTDYWIVKNSWGSLWGDHGYIRMARNRDNMCGIASAASVPVVEGFL

SEQ ID NO: 24 - protein, 326 aa, Fasciola hepatica
  MISC_FEATURE (1)..(326): cathepsin L5
MRLVILTLLIVGVFASNDDLWHQWKRIYNKEYNGADDDHRRNIWEQNVKHIQEHNLRHDLGLVTYKLGLNQFTDMTFEEFKAKYLTEMPRASELLSHGIPYKANKRAVPDRIDWRESGYVTEVKDQGGCGSCWAFSTTGAMEGQYMKNQRTSISFSEQQLVDCSRDFGNYGCNGGLMENAYEYLKRFGLETESSYPYRAVEGQCRYNEQLGVAKVTGYYTVHSGDEVELQNLVGAEGPAAVALDVESDFMMYRSGIYQSQTCSPDRLNHGVLAVGYGIQDGTDYWIVKNSWGTWWGEDGYIRMVRKRGNMCGIASLASVPMVAQFP

SEQ ID NO: 25 - protein, 306 aa, Fasciola hepatica
  MISC_FEATURE (1)..(306): cathepsin L6
WHEWKRMYNKEYNGADDEHRRNIWEQNVKHIEEHNLRHDLGLVTYKLGLNQFTDLTFEEFKAKYLMEMSPESESLSDGISYEAEGNDVPASIDWRQYGYVTEVKDQGGCGSCWAFSTTGAIEGQYVKKFQTRVSFSEQQLVDCSTIPGNHGCRGGGMRRAYEYLKKNGLEPESSYPYKAVEGQCQYKSDLALAKVTNSQLVRSGNETQLKNLIGAEGPASVAVDVKPDFSMYRSGIYQSQTCSSRRMNHAVLAVGYGTEGGMDYWIVKNSWGPRWGEAGYIRMARNRNNMCGIASAGSLPTVERFP

SEQ ID NO: 26 - protein, 337 aa, Fasciola hepatica
  MISC_FEATURE (1)..(337): cathepsin B
MSWLLIFAAIVVAQAKPNYKRQFEPFSDELIHYINEESGASWKAAPSTRFNNIDQVKQNLGVLEETPEDRNTQRQTVRYSVSENDLPESFDARQKWANCPSISEIRDQSSCSSCWAVSSASAITDRICIHSNGQKKPRLSAIDIVSCCAYCGYGCNGGIPAMSWDYWTREGVVTGGTLENPTGCLPYPFPKCSHGVVTPGLPPCPRDIYPTPKCEKKCHAGYNKTYEQDKVKGKSSYNVGGQETDIMMEIMKNGPVDGIFYMFEDFLVYKSGIYHYTTGRLVGGHAIRVIGWGVENGVKYWLIANSWNEGWGEKGYFRMRRGNNECGIEARINAGLP

SEQ ID NO: 27 - protein, 247 aa, Fasciola hepatica
  MISC_FEATURE (1)..(247): cathepsin B protease
RSQWPQCWTISEIRDQASCGSCWATAAASAMSDRVCIHSNGQMRPRLAAADPLSCCTYCGQGCRGGYPPKAWDYWMREGIVTGGTWENRTGCQPWMFTKCDHVGDSRKYSRCPHYTYPTPPCARACQTGYNKTYEQDKFYGNSSYNVGEHESYIMQEIMKNGPVEVTFAIFQDFGVYRSGIYHHVAGKFIGRHAVRMIGWGVENGVNYWLMANSWNEEWGENGYFRMVRGRNECGIESEVVAGMPRL

SEQ ID NO: 28 - protein, 112 aa, Fasciola hepatica
  MISC_FEATURE (1)..(112): putative cathepsin B
YEQDKVKGKSSYNVGEQETDIMMEIMKNGPVDGIFYMFEDFLVYKSGIYHYTTGRLVGGHAIRVIGWGVENGVKYWLIANSWNEGWGEKGYFRMRRGNNECGIEARINAGLP

SEQ ID NO: 29 - protein, 337 aa, Fasciola hepatica
  MISC_FEATURE (1)..(337): pro-cathepsin B
VVVQAAPNEKPQFEPFSDELIHYINEKSGASWKAAPSSRFINIEHFKQHLGLLEETPEERQTRRPTVRYNVSDNDLPESFDAREKWPLCRSIRQIPDQSSCGSCWAVAGVGAMSDRVCIHSNGMMQPELSAIDLVSCCSYCGNGCQGGSPPAAWDYWWRNGIVTGGTLENPTGCLPYPFPQCRHPGSRSQLNPCPRYTYPTPSCYPYCQAGYDKTYEKDKVYGKTSYNVDRHEYTIMEEIMKNGPVEAGFIVYTDFAVYKSGIYHHVSGRYAGKHAIRIIGWGVENGVKYWLTANSWNVGWGENGYFRILRGTDECRIESIVVAGMPRLQKNITNHH

SEQ ID NO: 30 - protein, 278 aa, Fasciola hepatica
  MISC_FEATURE (1)..(278): cathepsin B3
EPFSDELIHYINEESGASWKAAPSTRFNNIDQVKQNLGVLEETPEDRNTQRQTVRYSVSENDLPESFDARQKWPNCPSISEIRDQSSCSSCWAVSSASAITDRICIHSNGQKKPRLSAIDIVSCCAYCGYGCNGGIPAMSWDYWTREGVVTGGTLENPTGCLPYPFPKCSHGVVTPGLPPCPRDIYPTPKCEKKCHAGYNKTYEQDKVKGKSSYNVGEQETDIMMEIMKNGPVDGIFYMFEDFLVYKSGIYHYTTGRLVGGHAIRVIGWGVENGVNYW

SEQ ID NO: 31 - protein, 278 aa, Fasciola hepatica
  MISC_FEATURE (1)..(278): cathepsin B2
EPFSDELIRFVNEESGASWKAARSTRFSNVDHFKLDLGALSETPEERNALRPTIKHDISKNDLPESFDARSQWPQCWTISEIRDQASCGSCWATAAASAMSDRVCIHSNGQMRPRLAAADPLSCCTYCGQGCRGGYPPKAWDYWMREGIVTGGTWENRTGCQPWMFTKCDHVGDSRKYSRCPHYTYPKPPCARACQTGYNKTYEQDKFYGNSSYNVGEHESYIMQEIMKNGPVEVTFAIFQDFGVYRSGIYHHVAGKFIGRHAVRMIGWGVENGVNYW

SEQ ID NO: 32 - protein, 278 aa, Fasciola hepatica
  MISC_FEATURE (1)..(278): cathepsin B1
EPFSDELIHYINEKSGASWKAGPSSRFINIEHFKQHLGLLEETPEERETRRPTVRYNVSENDLPESFDAREKWPLCRSIRQIPDQSSCGSCWAVAGVGAMSDRVCIHSNGMMQPELSAIDLVSCCSYCGNGCQGGSPPAAWDYWWRNGIVTGGTLENPTGCLPYPFPQCRHPGSRSQLNPCPGYIYPTPSCYPYCQAGYDKTYEEDKVYGKTSYNVDRHEYTIMQEIMKNGPVEAGFIVYTDFAVYKSGIYHHVSGRYAGKHAIRIIGWGVENGVNYW

SEQ ID NO: 33 - protein, 194 aa, Fasciola hepatica
  MISC_FEATURE (1)..(194): peroxiredoxin
MLQPNMPAPNFSGQAVVGKEFETISLSDYKGKWVILAFYPLDFTFVCPTEIIAISDQMEQFAQRNCAVIFCSTDSVYSHLQWTKMDRKVGGIGQLNFPLLADKNMSVSRAFGVLDEEQGNTYRGNFLIDPKGVLRQITVNDDPVGRSVEEALRLLDAFIFHEEHGEVCPANWKPKSKTIVPTPDGSKAYFSSAN

SEQ ID NO: 34 - protein, 194 aa, Fasciola hepatica; identical to SEQ ID NO: 33
  MISC_FEATURE (1)..(194): peroxiredoxin

SEQ ID NO: 35 - protein, 218 aa, Fasciola hepatica
  MISC_FEATURE (1)..(218): peroxiredoxin
MCDRDQCSPGRHPLPHSHPHLQRPMLQPNMPAPNFSGQAVVGKEFKTISLSDYKGKWVILAFYPLDFTFVCPTEIIAFSDQMEQFARRNCAVIFCSTDSVYSHLQWTKMDRKVGGIGQLNFPLLADKNMSISRAYGVLDEEQGNTYRGNFLIDPKGVLRQITVNDRPVGRSVEEALRLLDAFIFHEEHGEVCPANWKPKSKTIVPTPDGSKAYFSSAN

SEQ ID NO: 36 - protein, 194 aa, Fasciola hepatica
  MISC_FEATURE (1)..(194): peroxiredoxin
MLQPNMPAPNFSGQAVVGKEFKTISLSDYKGKWVILAFYPLDFTFVCPTEIIAFSDQMEQFARRNCAVIFCSTDSVYSHLQWTKMDRKVGGIGQLNFPLLAEKNMSISRAYGVLDEEQGNTYRGNFLIDPKGVLPQITVNDRPVGRSVEEALRLLDAFIFHEEHGEVCPANWKPKSKTIVPTPDGSKAYFSSAN